(19) United States
(12) Patent Application Publication
(10) Pub. No.: US 2026/0294728 A1
(43) Pub. Date: Oct. 1, 2026

(54) System for Compressing a Part of a Living Being

(71) Applicant: SODIT, Levallois-Perret (FR)

(72) Inventor: Florian Desseree, Le Pont-de-Beau-Voisin (FR)

(73) Assignee: SODIT, Levallois-Perret (FR)

(21) Appl. No.: 19/479,441

(22) PCT Filed: Apr. 21, 2024
(86) PCT No.: PCT/EP2024/060852

§ 371 Date: Oct. 28, 2025

(30) Foreign Application Priority Data
Apr. 28, 2023 (FR) FR2304349

(51) Int. Cl. A61H 11/00

(52) U.S. Cl. CPC A61H 11/00; A61H 2011/005; A61H 2201/1215; A61H 2209/00

(57) ABSTRACT

The invention relates to an active module (M_0) that can be used in a system for compressing a part of a living being, said active module (M_0) comprising N compression modules (M_i) juxtaposed along a longitudinal axis (Y), N being greater than or equal to 2, each compression module (M_i) comprising at least one first traction member (30) secured to the first edge (200) of the active module (M_0) and means for driving said first traction member in translation, each compression module being electronically controllable to perform a clamping effect by translational displacement of its first traction member (30) along a clamping axis (X) perpendicular to said longitudinal axis (Y), said first traction member (30) of each compression module (M_i) being attached to at least one other first traction member (30) of an adjacent compression module via first connection means.

### TECHNICAL FIELD OF THE INVENTION

The present invention relates to a system for compressing a part of a living being.

### PRIOR ART

By way of example, the treatment of a pathology such as lymphedema is carried out using a compression system enveloping the limb that is to be treated. There are systems which are referred to as passive, such as those described in the patent application US2015/366720A1, and which use a simple sleeve with several clamping rings whose compression levels are adjusted manually by means of straps. There are also active treatment systems. Among the active systems, some use a pneumatic solution such as that described in the patent U.S. Pat. No. 6,406,445B1. The system is then composed of several rings juxtaposed along the sleeve, each ring being composed of a pneumatically controlled tube. The implementation of a sequence makes it possible to inflate one or more tubes in a manner suitable for circulating the lymphatic fluid.

Among the active systems, there are also those which use shape-memory alloy actuators distributed along the sleeve. A wire made of this alloy retracts upon heating. By controlling each actuator individually, it is thus possible to apply localized compression. Such active compression systems are described, for example, in the patent applications US2016/074234A1 and US2017/304136A1, and also in the patent U.S. Pat. No. 5,997,465. The latter patent, U.S. Pat. No. 5,997,465, establishes the principle of using shape-memory alloys in compression systems. The patent application US2016/074234A1 proposes, in addition to the principle of compression with shape-memory alloy actuators, the concept of providing thermal energy in order to improve the treatment performed by the system. For its part, the patent application US2016/074234A1 proposes mechanical solutions for locking the actuator in the retracted position.

Similar solutions are also described in the prior art documents DE102008003124A1, US015/073318A1, US2018/242655A1 and US2018/177677A1.

For its part, the patent application WO2020/144437A1 describes a solution also using several juxtaposed clamping/unclamping units, each unit comprising a plate that is cut out in a suitable way to give it a spring function, the plate being inserted between two free edges of the sleeve. A wire made of a shape-memory alloy is electrically energized to retract and actuate the plate and to allow compression of the limb.

It has been found that these prior art solutions are not all optimal, whether in terms of compactness, the ratio of compactness to efficiency, the ease of use or the ease of manufacture.

In addition, the use of shape-memory alloy wires has certain drawbacks:
It is not possible to keep the system in the retracted position without maintaining electrical power or using a complex locking mechanism;
The wires tend to heat up, requiring special insulation and/or making it necessary to control the compression level in order to limit heating;
The battery required for power supply must have a relatively large capacity to ensure sufficient battery life.

The patent application EP1013220A1 describes an apparatus for measuring blood flow, for its part using an electric motor actuated to clamp a ring, via a pulling linkage wound around a pulley.

This prior principle using a motor and a linkage has been used in lymphatic drainage solutions, as described in the patent application US2021/121356A1. In this patent application, the device uses a simple wire pulled by a motor, the wire passing around a return member. The pulling of the wire has the effect that two edges of a sleeve placed around the limb to be treated are brought closer together, thus allowing localized compression of the limb. In this prior art document, however, the proposed solution is complex to manufacture, not especially user-friendly and not very practical to use by the user.

Moreover, it has been found that the prior art solutions were not always suitable for ensuring homogeneous compression over the entire length of the limb when the limb has a non-constant cross section. For example, a limb such as a forearm has a somewhat frustoconical cross section, with a larger cross section at the top and a smaller cross section at the bottom. In the prior art, the known pneumatic pressotherapy solutions use several juxtaposed tubes, each of them controlled independently in order to provide an identical pressure over the entire width of the tube. In other words, this type of solution does not take into account the variation in the cross section of the limb. The pressure exerted by the tube will in fact be greater where the cross section of the limb is at its greatest, and less where the cross section of the limb is at its smallest.

There is therefore a need to obtain a compression device capable of ensuring homogeneous annular compression movements which follow the variations in cross section of the limb along its length.

### DESCRIPTION OF THE INVENTION

This object is achieved by an active module that can be used in a system for compressing a part of a living being, said active module comprising N compression modules juxtaposed along a longitudinal axis between two opposite edges, called the first edge and the second edge of said active module, N being greater than or equal to 2 and each compression module being defined by a rank i, with i ranging from 1 to N, each compression module comprising at least one first traction member secured to the first edge of the active module and means for driving said first traction member in translation, each compression module being electronically controllable to perform a clamping effect by translational displacement of its first traction member along a clamping axis perpendicular to said longitudinal axis, said first traction member of each compression module being attached to at least one other first traction member of an adjacent compression module via first connection means, said first connection means being arranged to effect a mechanical transfer of movement from the first traction member of each compression module to the first traction member of the adjacent compression module.

The principle of the invention makes it possible ultimately to obtain, over the entire system, a frustoconical compression profile having a substantial tangential component. This point is fundamental for the efficacy of the system and constitutes a notable difference compared to the existing solutions operating by pneumatic pressotherapy, in which the pressure is exerted uniformly over the entire width of the ring/tube.

According to a special feature of the active module:
The first traction member of the compression module of rank 1 is connected only to the first traction member of the compression module of rank 2,
The first traction member of the compression module of rank N is connected only to the first traction member of the compression module of rank N−1,
The first traction member of the compression module of rank i, for i ranging from 2 to N−1, is connected both to the first traction member of the compression module of rank i+1 and to the first traction member of the compression module of rank i−1.

Advantageously, the first connection means are non-elastic.

According to a particular embodiment, the first connection means are designed as a mechanical pivoting connection about an axis perpendicular to the longitudinal axis and to the clamping axis, in such a way that each first traction member forms the link of a chain.

According to another particular embodiment, the first connection means are implemented in the form of a linkage.

According to another particular embodiment, the first traction members of the compression modules are designed as a single piece.

Advantageously, each compression module comprises a second traction member secured to the second edge of the active module, the second traction member being able to be actuated in translation along said clamping axis.

According to one feature, said second traction member of each compression module is attached to at least one other second traction member of an adjacent compression module via second connection means, said second connection means being arranged to effect a mechanical transfer of movement from the second traction member of each compression module to the second traction member of the adjacent compression module.

According to another feature:
The second traction member of the compression module of rank 1 is connected only to the second traction member of the compression module of rank 2,
The second traction member of the compression module of rank N is connected only to the second traction member of the compression module of rank N−1,
The second traction member of the compression module of rank i, for i ranging from 2 to N−1, is connected both to the second traction member of the compression module of rank i+1 and to the second traction member of the compression module of rank i−1.

Advantageously, the second connection means are non-elastic.

According to a particular embodiment, the second connection means are designed as a mechanical pivoting connection about an axis perpendicular to the longitudinal axis and to the clamping axis, in such a way that each second traction member forms the link of a chain.

According to another particular embodiment, the second connection means are implemented in the form of a linkage.

According to another particular embodiment, the second traction members of the compression modules are designed as a single piece.

According to one particular feature, each compression module has at least one electric motor with an output shaft, and a pulling linkage secured to said output shaft of the electric motor and hooked on the one hand onto the first traction member and on the other hand onto the second traction member.

According to another particular feature, the pulling linkage is hooked onto the first traction member and onto the second traction member at a hooking point that is off-center with respect to the center of gravity of the first traction member and the second traction member, respectively.

The invention also relates to a system for compressing a part of the body of a living being, comprising a sleeve to be fitted around said part of the body of the living being, said sleeve comprising at least one piece made of textile material, to be positioned at least partially around said part of the body of the living being, and an active module, and means for securing said active module between two separate zones of said piece of textile material so as to effect a join between said two separate zones, characterized in that said active module is as defined above.

### BRIEF DESCRIPTION OF THE FIGURES

Other features and advantages will become apparent from the following detailed description given with reference to the appended drawings, in which:

FIG. 1 illustrates the principle of the compression system of the invention;

FIG. 2 shows schematically the principle of the architecture of the compression system of the invention;

FIG. 3 is an exploded view of the two parts of the compression system of the invention;

FIG. 4 shows the assembled compression system;

FIG. 5 shows an exemplary embodiment of the active module used in the compression system of the invention;

FIG. 6 shows the architecture of a compression module used in the compression system of the invention;

FIG. 7 shows the operating principle of the active module and of its juxtaposed compression modules;

FIG. 8A shows a first example of the connection of two traction members used in the active module of the compression system of the invention;

FIG. 8B shows a second example of the connection of two traction members used in the active module of the compression system of the invention;

FIG. 8C shows a third example of the connection of two traction members used in the active module of the compression system of the invention.

### DETAILED DESCRIPTION OF AT LEAST ONE EMBODIMENT

### Compression System—FIG. 1

The invention relates to a compression system 1 for performing localized compression effects along a part P of the body of a living being, for example a human being. By way of example, the part P of the body of a living being may be a limb but also any other part, for example the chest or the trunk. In a non-limiting manner, the limb to be treated may be an arm, a leg, a forearm, etc., of a living being.

In a non-limiting manner, the system 1 is in particular perfectly adapted to treat a pathology of the lymphedema type and to activate the flow of lymphatic fluid in the limb that is to be treated. As another example, it can also stimulate venous and lymphatic flows in order to facilitate recovery after sports activity.

In the remainder of the description, the compression system 1 of the invention is described to fit on a limb of a living being.

The compression system 1 is in the form of a sleeve that is intended to be positioned around the limb to be treated and that is to be adjusted around the latter. The sleeve is defined by its longitudinal axis, labeled (A1), and, when placed and adjusted around the limb, allows localized compression effects to be exerted in radial directions along the limb to be treated.

The sleeve advantageously comprises several parts described below.

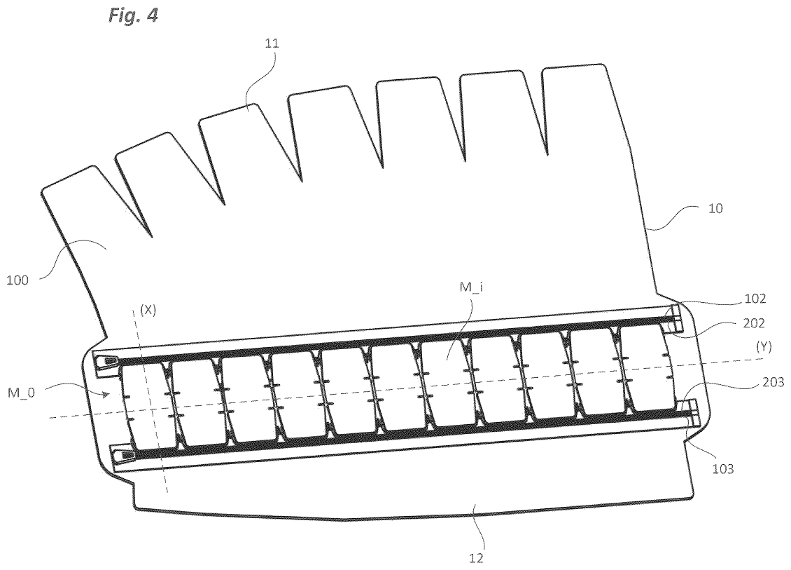

### First Part of the Sleeve: Textile Part—FIG. 3, FIG. 4

The sleeve comprises a first part 10 made of a flexible, advantageously non-elastic material, such as a textile, which is intended to be placed around the limb and which has a first free edge 11 and a second free edge 12. The first free edge 11 and the second free edge 12 can comprise adjustable and complementary temporary fastening means, such as touch-close strips, permanent magnets, adjustable straps, hooks, etc. The two free edges are joined to each other by these temporary fastening means so as to close and hold the sleeve around the limb to be treated. The temporary fastening means are advantageously chosen to enable the size of the sleeve to be adjusted to the size of the limb to be treated. FIG. 3 and FIG. 4 show this embodiment principle, the two free edges 11, 12 being provided here with complementary touch-close strips.

In the appended figures, the first part 10 is made in the form of a single textile piece, cut to the desired shape and size. It should be understood that it could be made as several pieces joined together permanently, in the form of pieces sewn together, or temporarily, by suitable fixing solutions (hooks, touch-close strips, etc.). The textile piece comprises two opposite faces, namely a first face, called the inner face 100, intended to be oriented toward the surface of the limb to be treated, and a second face, called the outer face 101, facing outward. This first part 10 advantageously goes all the way around the limb to be treated.

The first part 10 of the sleeve can be made of a textile material, for example a synthetic textile. This textile is advantageously not very extensible (deformation in extension of 10% maximum) or even non-extensible in order to effectively transmit the compression force created by the system.

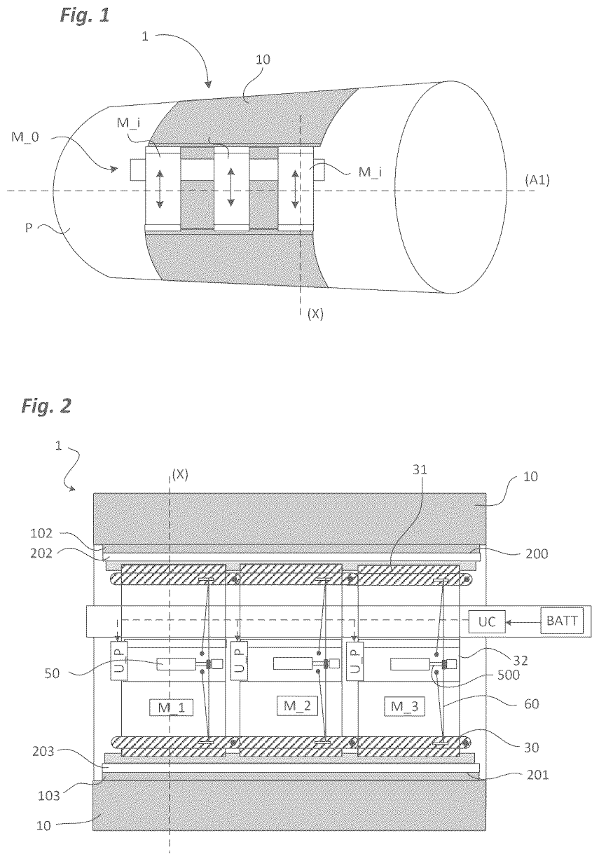

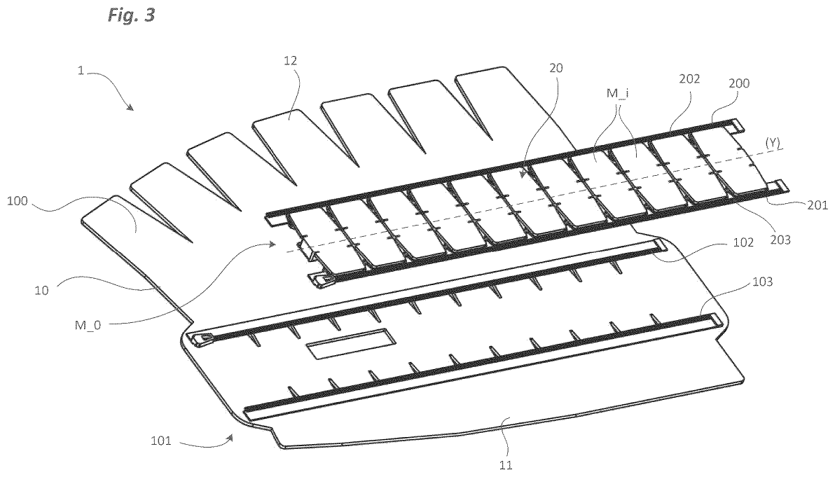

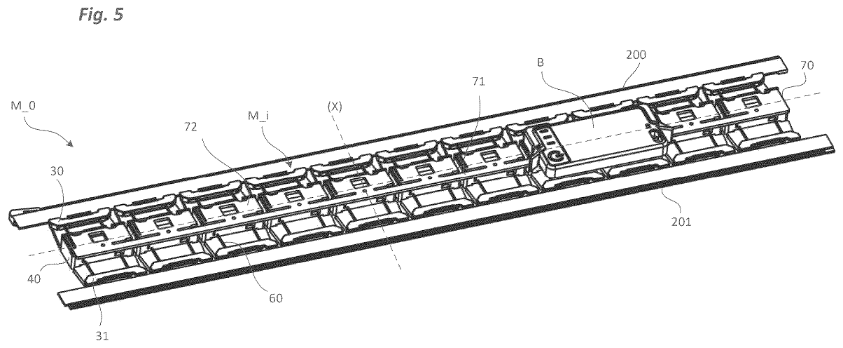

### Second Part of the Sleeve: Active Module—FIG. 2, FIG. 3, FIG. 4, FIG. 5

The sleeve comprises a second part 20 composed of the active module M_0 of the system.

The active module M_0 carries several compression modules M_i, advantageously all identical, juxtaposed along a second longitudinal axis (Y) parallel to the longitudinal axis (A1) of the sleeve. Each of these compression modules is configured to exert traction along an axis perpendicular to said second longitudinal axis (Y) and said clamping axis (X).

The active module M_0 of the system 1 is in the form of a single assembly. It is adapted to be assembled to the first part 10 of the system.

The compression modules are aligned along the active module in such a way as to form a series of compression modules. To simplify and better understand this, it can be considered that the active module comprises N juxtaposed compression modules, with N greater than or equal to 2. Each compression module is, for example, defined by a rank i in the series, with i ranging from 1 to N. The compression module of rank 1 and the compression module of rank N are therefore the two end compression modules. In general, the compression module will be designated M_i.

For the remainder of the description, a sequence of actuation of the compression modules is defined going from the module of rank 1, which is activated first in the sequence, to the module of rank N, which will be activated last in the sequence.

Each compression module M_i advantageously defines a distinct compression ring capable of exerting localized compression on a part of the limb to be treated.

The active module M_0 comprises at least two juxtaposed compression modules. Of course, the number of juxtaposed compression modules will be adapted to the length of the limb to be treated.

In a non-limiting manner, the active module M_0 can advantageously comprise temporary fastening means comprising a first band 202 of first temporary fastening means intended to cooperate with a second band 102 complementary to these first temporary fastening means and carried by the first part 10 of the sleeve, and a second band 203 of second temporary fastening means intended to cooperate with a second band 103 complementary to these second temporary fastening means and also present on the first part 10 of the system. According to a particular aspect of the invention, it thus has the advantage of being entirely detachable from the first part 10 of the system. It can be rectangular in shape, having two parallel opposite edges 200, 201, spaced apart and each provided with the respective first band 202, 203 of the first temporary fastening means and of the second temporary fastening means.

It should be noted that the principle of assembly of the active module M_0 on the first textile part 10 of the sleeve, via the temporary fastening means, is optional and that it could be held on this first part permanently.

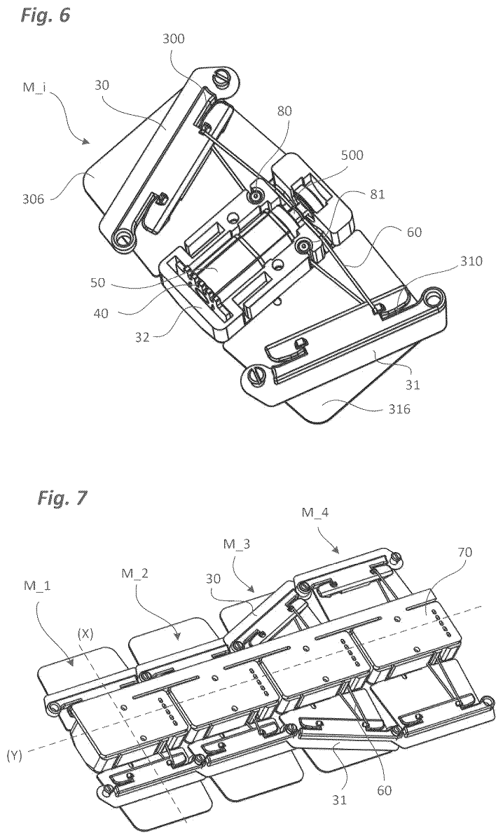

### Compression Module—FIG. 6

Each compression module M_i can be controlled to bring closer the two portions of the first part 10 of the sleeve to which it is attached, thus providing clamping and localized compression in a ring shape when the sleeve is placed around the limb to be treated.

Each compression module M_i can be implemented independently and can be removable with respect to the rest of the active module M_0. It is intended to provide a mechanical action and is advantageously controlled and supplied by an electronic control and supply circuit belonging to the system 1 and advantageously arranged on a support common to all the compression modules. The control circuit will be detailed below.

In a non-limiting manner, a compression module M_i comprises at least one traction member 30 and advantageously two traction members 30, 31, designated first traction member 30 and second traction member 31, which are separated from each other and are able to move in translation with respect to each other along the clamping axis (X). The relative positions of the first traction member 30 and of the second traction member 31 make it possible to adjust the level of clamping applied by the compression module M_1.

The first traction member 30 and the second traction member 31 are each made in the form of a separate piece, for example made of plastic material.

The first traction member 30 and the second traction member 31 are each in the form of a bar, for example. They are advantageously of identical shape. To simplify manufacture and assembly, each traction member has, for example, an architecture that is symmetrical with respect to the clamping axis (X).

The first traction member 30 is secured to the first edge 200 of the active module M_0, for example the one carrying the first band 202 of the first temporary fastening means. The securing can be effected by any suitable means.

The second traction member 31 is secured to the second edge 201 of the active module M_0, opposite the first edge and for example carrying the first band 203 of the second temporary fastening means. The securing can be effected by any suitable means.

Thus, when the active module M_0 is attached to the first part 10 of the sleeve, via the first and second temporary fastening means described above (when they are present), a traction on the first traction member 30 and on the second traction member 31 along the axis (X) makes it possible to bring the two opposite edges 200, 201 of the active module M_0 closer together and therefore to pull, in opposite directions along the axis (X), on the two portions of the sleeve to which these two edges are hooked, and thus to exert clamping and compression of the limb when the sleeve is fitted around the limb. Since the first part 10 of the sleeve is made of a textile material that is not very extensible or even non-extensible, the clamping force exerted by each compression module M_i is transmitted directly to the sleeve in order to effect compression of the limb.

The first traction member 30 and the second traction member 31 can each have a body, for example made of plastic material.

The compression module M_i can advantageously comprise a support housing 32.

The compression module M_1 comprises an electric motor 50 which is accommodated in said support housing 32.

The electric motor 50 comprises an output shaft 500.

The compression module M_i also comprises a pulling linkage 60 which is wound or unwound, by the action of the electric motor 50, on the output shaft 500, in order to ensure the displacement of the first traction member 30 and of the second traction member 31.

Advantageously, the compression module comprises a single pulling linkage in order to ensure the simultaneous displacement of the two traction members 30, 31.

The pulling linkage 60 can be a string, a fine cable or equivalent. It is secured, for example, to the output shaft 500 of the electric motor 50, for example with the aid of a plastic part force-fitted onto this output shaft 500.

By way of example, at a first end, the pulling linkage 60 is secured to a fixed point 80 formed on the support housing 32, the securing being effected, for example, with the aid of a first crimped aluminum part. The pulling linkage 60 then extends toward the first traction member 30 and passes through a slot 300 made on the first traction member 30 in order to hook onto the latter. Then, on its return path toward the second traction member 31, the pulling linkage 60 is secured around the output shaft 500 of the electric motor 50. It is then passed through a slot 310 of the second traction member 31 in order to hook onto the latter. It is then returned to a second fixed point 81 (advantageously separate from the first fixed point), on which its second end is hooked, formed on the support housing 32, the securing being effected, for example, with the aid of a second crimped aluminum part.

On each traction member, the pulling linkage 60 advantageously comprises only one hooking point, this hooking point being positioned off-center so as to be able to tilt the traction member (see operating principle below).

The hooking point of the pulling linkage 60 on each traction member is advantageously identical for all the compression modules. For each compression module M_i, the hooking point on the first traction member 30 and the hooking point on the second traction member 31 are advantageously positioned symmetrically with respect to the longitudinal axis (Y). The same applies to the first fixed point 80 and the second fixed point 81.

Advantageously, in their translational movement, each traction member is caused to slide on a support panel 306, 316, advantageously made of a plastic material, advantageously flexible in order to match the morphology of the limb to be treated.

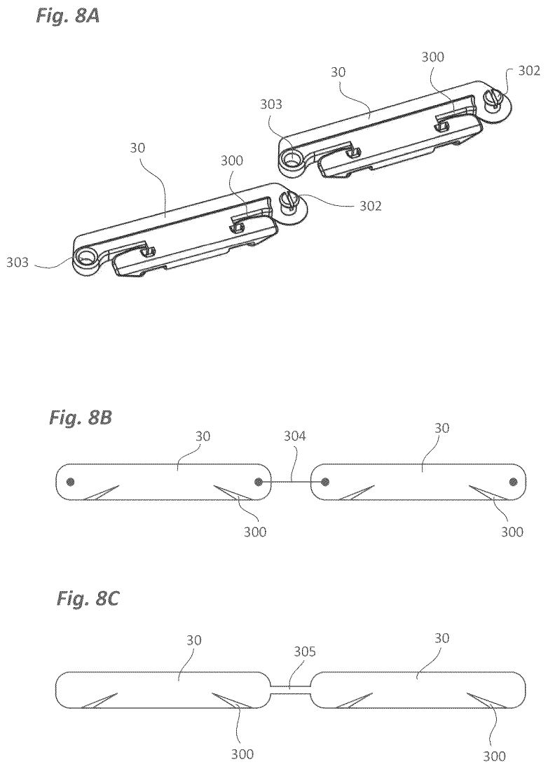

### Connection of the Traction Members—FIG. 7, FIG. 8A, FIG. 8B, FIG. 8C

The principle of the invention is to connect the traction members (first connection between the first traction members on the one hand and second connection between the second traction members on the other hand) to each other in order to effect a transfer of the mechanical movement of each traction member to at least one adjacent traction member in the series. The connection between two adjacent traction members is therefore made in such a way as to be able to effect this transfer of mechanical movement. It is therefore a question of connecting each traction member to at least one other adjacent traction member (or to two traction members depending on its position in the series of compression modules) by way of connection means, such that, during the pulling movement of a traction member, the latter drives at least partially the movement of the adjacent traction member (see operating principle below). The connection means connect the two traction members directly via their respective movable part, which can be actuated by the motor of the compression module via the pulling linkage. The term “directly” is understood to mean that the connection is made by at least one mechanical element between the two traction members, in order to connect a securing point present on the first traction member to a securing point present on the second traction member, without passing through the rest of the body of the compression module.

The traction members are therefore interconnected so as to form a chain with several links, each link being represented by a separate traction member. The device thus comprises two chains arranged in parallel, each coming together on a separate edge.

According to the invention, the first traction member 30 of each compression module M_i is attached to at least one other first traction member 30 of an adjacent compression module (M_i+1, M_i−1 or both).

According to the invention, this principle is also applied to the second traction member 31 of each compression module M_i. Thus, the second traction member 31 of each compression module M_i is attached to at least one other second traction member 31 of an adjacent compression module (M_i+1, M_i−1 or both).

Of course, it should be noted that the first traction member 30 of the compression module of rank 1 and that of the compression module of rank N are each attached only to the first traction member of a single compression module, respectively the one which follows it (M_i+1) or precedes it (M_i−1) directly in the series of compression modules.

The first traction member 30 of each compression module M_i, for i ranging from 2 to N−1, is connected both to the first traction member 30 of the compression module placed upstream in the series and to the first traction member 30 of the compression module placed downstream in the series.

The first traction member of each compression module of rank i comprises a first attachment point for attaching to the first traction member of the compression module of rank i−1 and a second attachment point for attaching to the first traction member of the compression module of rank i+1.

Similarly, the second traction member 31 of the compression module of rank 1 and that of the compression module of rank N are each attached only to the second traction member 31 of a single compression module, respectively the one which follows it or precedes it directly in the series of compression modules.

The second traction member 31 of each compression module M_i, for i ranging from 2 to N−1, is connected both to the second traction member 31 of the compression module placed upstream in the series and to the second traction member 31 of the compression module placed downstream in the series.

According to the invention, the connection between two traction members of two successive compression modules is achieved by using connection means that are advantageously virtually non-elastic, and even totally non-elastic, in order to be able to better transfer the mechanical movements between them.

These connection means can be made according to different alternative embodiments described below.

According to a first variant embodiment shown in FIG. 8A, the connection means are non-elastic and comprise an assembly device forming a pivot connection between two adjacent traction members 30, so that each traction member 30 thus forms the link of a chain.

The pivot connection is achieved by providing the link with a male part 302 cooperating in pivoting connection with a female part 303 of the adjacent link.

According to a second variant embodiment shown in FIG. 8B, the connection means are advantageously non-elastic and comprise a linkage 304, such as a string or equivalent, connecting two adjacent traction members 30.

According to a third variant embodiment shown in FIG. 8C, the traction members of the compression modules are joined together to form a single piece made of single material or bi-injected material. This piece 305 thus comprises the traction members 30 of all the compression modules of the active module.

The principles described above with reference to FIGS. 8A, 8B and 8C apply identically to the two traction members 30, 31 of each compression module M_i.

According to a particular aspect of the invention, the hooking point of the pulling linkage 60 on each traction member (corresponding to the location of the hooking slot 300, 310 on the traction member) of a module of rank i is situated as close as possible to its first point of attachment to the traction member of the compression module of rank i+1. It is thus off-center with respect to the center of gravity of the traction member. In the case of the first variant embodiment (FIG. 8A), the pulling linkage 60 is therefore hooked as close as possible to the pivot connection formed between two links of the chain, on the side of its point of attachment to the corresponding traction member of the compression module of rank i+1.

### Operating Principle of the Active Module—FIG. 7

In operation, starting from an assembly according to the first variant embodiment of FIG. 8A, when a compression module M_2 is activated, the ring compresses by pulling on its two traction members 30, 31, by virtue of the pulling linkage 60. In its compression movement, each traction member 30, 31 drives with it the traction member of the adjacent compression module M_3 in the series, via their mechanical connection (for example the pivoting connection). In this situation, the two traction members 30, 31 of the compression module M_2, which are activated and completely retracted, and the two traction members 30, 31 of the adjacent compression module M_3, which are inclined with respect to a direction parallel to the longitudinal axis (Y), are driven by virtue of the mechanical connection. This principle continues with each new activation of a compression module, making it possible to follow the variation in cross section of the limb, in particular when the latter has a frustoconical shape. It can be seen in FIG. 7 that the compression module M_3 is therefore not yet activated, its pulling linkage 60 being still deployed. However, its traction members 30, 31 are pivoted inward, each of them driven by the corresponding traction member of the compression module M_2 actuated upstream. For its part, the module M_4 is not yet activated. Its traction members 30, 31 are still in the rest position, parallel to the longitudinal axis (Y).

According to the invention, the compression generated by each compression ring is therefore not homogeneous over the entire width of the ring but has a frustoconical pressure profile. The generated pressure has a high tangential component along the longitudinal axis Y, which directs the migration of the body fluids (blood and lymph). In a non-limiting manner, the system 1 is, for example, oriented so as to promote migration from the distal part toward the proximal part of the limb. In the event that the reverse direction is necessary (for example to bring blood toward the extremities), it would be sufficient to turn the system around.

It should be noted that it would be possible to divide the compression modules into several distinct groups, each group being able to comprise one or more compression modules. In each group which comprises several adjacent compression modules, the traction members of these compression modules are connected according to one of the principles of embodiment of the connection means.

### Compression Module Control Circuit—FIG. 2, FIG. 5

Each compression module M_i can comprise a dedicated control unit U_P. The control unit U_P is of the electronic type and can comprise a first electrical terminal and a second electrical terminal to which the electric motor 50 of the compression module M_1 controlled by the control unit U_P is connected. This control unit U_P is intended to control the electric motor 50 of the compression module M_i, for example in order to control its activation, the direction of rotation of the motor 50 and its speed of rotation. This control unit U_P can be made on an electronic control board 40 that is accommodated vertically in the support housing 32 of the module. The electric motor 50 is connected to this electronic control board 40 by soldering or by tight insertion of its connections. In addition, the electronic control board 40 comprising the control unit U_P comprises several upright “pins”, enabling it to be connected and soldered to the control and supply circuit common to all the compression modules of the system.

This control and power supply circuit can be produced according to different alternative embodiments.

Advantageously, the control and supply circuit is common to all the compression modules M_1 of the system and forms part of the active module M_0. It can be produced on an independent electronic control and power supply board 70. This board can extend in a direction parallel to the longitudinal axis (Y), over all the modules of the system, which makes it possible to connect their control unit U_P. The board 70 carries the control unit UC and possibly a battery block BATT, for example assembled in the same control and power supply unit B. Of course, other alternative embodiments could be envisioned. In a non-limiting manner, the control and power supply block B is removable and fixed to the electronic control and power supply board 70 by a clipping system. The electronic connection is provided by spring contacts, for example. This allows the control and power supply block B to be changed in the event of battery discharge or of an update/improvement of the control and power supply block B to a version having more options, for example for a control and power supply block B having a touch screen control or wireless connectivity. The electronic control and power supply board 70 may carry no electronic component and serve only to connect the block to the various control units U_P.

Advantageously, the board 70 is cut out to form several juxtaposed covers 72 connected to each other and each closing, at least partially, the receiving seat formed by the support housing 32 of the motor 50 of each compression module M_i.

The board 70 is also advantageously cut out in a suitable manner to form a plurality of connecting branches 71, each providing the junction between two juxtaposed covers 72 and therefore between two juxtaposed compression modules. Two juxtaposed covers 72 are, for example, connected by a single connecting branch 71. Each connecting branch 71 advantageously has mechanical characteristics of deformability, particularly in bending, at least about an axis perpendicular to the plane of the board 70. The flexibility of each branch 71 makes it possible to adapt the shape of the system to the level of compression applied by each module. In other words, the compression modules M_1 of the same system 1 do not necessarily all apply the same compression level; the flexibility of the board 70 makes it possible to compensate for the variations in compression levels applied from one compression module to another.

This board 70 is advantageously made of a composite material used for producing electronic circuits.

The compression system of the invention thus has many advantages, among which:
Each compression module operates with a motor 50 and a simple pulling linkage 60 hooked onto two traction members 30, 31, which are located on either side of its support housing 32. The traction members are interconnected by connection means (pivot points, for example) making it possible to adapt to the shape of the limb, in particular when the latter has a frustoconical shape. The mechanical movement of the traction member causes the mechanical movement of at least one adjacent traction member.
The compression modules are connected by the electronic control and power supply board, which is flexible and provides the electrical, electronic and mechanical connection. This principle allows simple and inexpensive assembly. The board is cut to provide flexibility about the three axes in order to conform the mechanism to morphological variations.
The active module is advantageously attached to the textile part of the sleeve via temporary fastening means (e.g. a zipper).

1. An active module (M_0) that can be used in a system for compressing a part of a living being, said active module (M_0) comprising N compression modules (M_i) juxtaposed along a longitudinal axis (Y) between two opposite edges (200, 201), wherein the opposite edges comprise a the first edge (200) and a second edge (201) of said active module, N being greater than or equal to 2, wherein each compression module-is defined by a rank i, with i ranging from 1 to N, wherein each compression module (M_i) comprises at least one first traction member (30) secured to the first edge (200) of the active module (M_0) and means for driving said first traction member in translation, wherein each compression module is electronically controllable to perform clamping by translational displacement of its first traction member (30) along a clamping axis (X) perpendicular to said longitudinal axis (Y), wherein said first traction member (30) of each compression module (M_i) is attached to at least one other first traction member (30) of an adjacent compression module via first connection means, said first connection means being arranged to effect a mechanical transfer of movement from the first traction member (30) of each compression module (M_i) to the first traction member of the adjacent compression module.

2. The active module (M_0) as claimed in claim 1, wherein the first traction member (30) of the compression module of rank 1 is connected only to the first traction member of the compression module of rank 2, wherein the first traction member (30) of the compression module of rank N is connected only to the first traction member of the compression module of rank N−1, and wherein the first traction member (30) of the compression module of rank i, for i ranging from 2 to N−1, is connected both to the first traction member of the compression module of rank i+1 and to the first traction member of the compression module of rank i−1.

3. The active module as claimed in claim 1, wherein the first connection means is non-elastic.

4. The active module as claimed in claim 3, wherein the first connection means comprises a mechanical pivoting connection about an axis that is perpendicular to both the longitudinal axis (Y) and to the clamping axis (X), wherein each first traction member forms the link of a chain.

5. The active module as claimed in claim 3, wherein the first connection means are implemented in the form of a linkage.

6. The active module as claimed in claim 3, wherein each of the first traction members of the compression modules is designed as a single piece.

7. The active module as claimed in claim 1, wherein each compression module (M_i) comprises a second traction member (31) that is secured to the second edge (201) of the active module (M_0) and wherein the second traction member can be actuated to translate along said clamping axis (X).

8. The active module as claimed in claim 7, wherein said second traction member (31) of each compression module (M_i) is attached to at least one other second traction member (10) of an adjacent compression module via second connection means, said second connection means being arranged to effect a mechanical transfer of movement from the second traction member (31) of each compression module (M_i) to the second traction member of the adjacent compression module.

9. The active module (M_0) as claimed in claim 8, wherein: the second traction member (31) of the compression module of rank 1 is connected only to the second traction member of the compression module of rank 2, wherein the second traction member (31) of the compression module of rank N is connected only to the second traction member of the compression module of rank N−1, and wherein the second traction member (31) of the compression module of rank i, for i ranging from 2 to N−1, is connected both to the second traction member of the compression module of rank i+1 and to the second traction member of the compression module of rank i−1.

10. The active module as claimed in claim 8, wherein the second connection means is non-elastic.

11. The active module as claimed in claim 10, wherein the second connection means comprises mechanical pivoting connection about an axis perpendicular both to the longitudinal axis (Y) and to the clamping axis (X) such that each second traction member (31) forms the link of a chain.

12. The active module as claimed in claim 10, wherein the second connection means are implemented in the form of a linkage.

13. The active module as claimed in claim 10, wherein the second traction members (31) of the juxtaposed compression modules are designed as a single piece.

14. The active module as claimed in claim 7, wherein each compression module (M_i) has at least one electric motor (50) with an output shaft (500), and a pulling linkage (60) secured to said output shaft of the electric motor and hooked on the one hand onto the first traction member (30) and on the other hand onto the second traction member (31).

15. The active module as claimed in claim 14, wherein the pulling linkage (60) is hooked onto the first traction member (30) and onto the second traction member (31) at a hooking point that is off-center with respect to the center of gravity of the first traction member and the second traction member, respectively.

16. A system for compressing a part of the body of a living being, said system comprising a sleeve to be fitted around said part of the body of the living being, said sleeve comprising at least one piece made of textile material, which is to be positioned at least partially around said part of the body of the living being, an active module (M_0), and means for securing said active module (M_0) between two separate zones of said piece of textile material so as to effect a joint between said two separate zones, wherein said active module (M_0) is as defined in claim 1.
